(19) United States
(12) Patent Application Publication
(10) Pub. No.: US 2004/0126444 A1
(43) Pub. Date: Jul. 1, 2004

(54) Cactaceae-based formulation having the property of fixing fats and method for obtaining same

(76) Inventors: Jean-Baptiste D'Huart, Carcassone (FR); Constantin Dallas, Beziers (FR)

Correspondence Address: Young & Thompson, Second Floor, 745 South 23rd Street, Arlington, VA, 22202, US

(21) Appl. No.: 10/474,455

(22) PCT Filed: Apr. 10, 2002
(86) PCT No.: PCT/FR02/01254

(30) Foreign Application Priority Data
Apr. 11, 2001 (FR) 01/04960

(51) Int. Cl. A61K 35/78

(52) U.S. Cl. 424/767

(57) ABSTRACT

The invention concerns a preparation having the property of fixing fats (so as to eliminate from digestion part of the fats ingested during a meal), based on cladodes of cactaceae of the edible type and at least partly non-available. The invention is characterised in that the cactaceae is in the form of powder particles whereof at least 70 wt. % have a size less than 100 μm (sieve-controlled particle size distribution). Advantageously, the inventive particles of cactaceae cladodes are in the form of granules coated with an acid-resistant film soluble in the intestine and/or with prolonged release. The invention also concerns a method for obtaining such a preparation.

The invention relates to a process for obtaining a cactus (cactacea)-based preparation, which is capable of fixing fats and which is intended to be ingested, for example in the context of a slimming diet. The invention also relates to a preparation obtained by this process, and to its uses.

Chitosan, derived from chitin contained especially in the cell walls of certain plants, bacteria or crustaceans, is a non-assimilable product having the property of fixing fats. It is used in man by ingestion, as an accompaniment to slimming diets, to allow the ingested fats to be subtracted from digestion. Despite acknowledged efficacy, this product is at the present time controversial.

It is consequently desirable to find novel products with identical if not better properties, which satisfy the requirements of nutritionists.

The cladodes of certain cacti are consumed by certain populations of South America and Central America as cooked vegetables or raw vegetables in salads. These foods are appreciated, among other reasons, for their supply of water and carbohydrates; they are also consumed as diuretic, laxative, antispasmodic medicinal plants or for, treating fevers, ulcers or hyperglycaemia. In particular, Opuntia Ficus Indica—commonly known as “nopal”—which is cultivated in Mexico, is consumed in the context of diets for treating obesity, hyperlipidaemia and diabetes. Studies [see “Effects of Nopal (Opuntia) on serum lipids, glycaemia and body weight”, Mexican archives of medical research, Vol. 14, No. 2, 1983] conducted with obese, diabetic and healthy individuals, showed that the ingestion of nopal cladodes for several days before the three main meals made it possible to significantly reduce their level of LDL (Low Density Lipoprotein, known as “bad cholesterol”), and also to reduce the level of triglycerides and the weight of the obese and diabetic individuals, as well as the glycaemia of the diabetics (a spreading-out over time of the assimilation of sugars is moreover observed). It is for this reason that cactus cladodes are introduced into the composition of certain therapeutic products intended for diabetics (see XP-002189940, Zhejiang Prov. People's Hospital) or are prescribed in the case of obesity, “bad cholesterol” or digestive disorders (see XP-002189939, Iwasaki S).

However, in vitro tests performed by the inventors (“primary fixing test” described later) have shown that the fat-fixing capacity of nopal cladodes as sold commercially for consumption is appreciably lower than that of chitosan.

The invention is directed towards proposing a product, and a process for obtaining such a product, having the property of fixing fats in proportions at least equivalent to those of chitosan. The invention is directed towards proposing a product that may be ingested without risk (i.e. that shows no harmfulness) and that has the property of fixing fats in vivo, for the purpose of subtracting them from digestion.

Another object of the invention is to use natural products such as cactus cladodes, and to substantially improve their properties without chemical conversion or addition.

The invention is moreover directed towards achieving all these objects simply and economically.

To this end, the invention is directed towards a preparation having the property of fixing fats, based on cladodes of a cactacea plant of edible and at least partially non-assimilable type, wherein the cactacea plant is in the form of a powder of particles, at least 70% by weight of which are smaller than 100 μm. Throughout the text hereinbelow, the expression “particles smaller than a given value” means: particles that have passed through, during a screening process, a screen having square mesh apertures with a side length equal to the said value. Moreover, the expression “cactacea plant of edible type” means a non-toxic cactacea plant, i.e. a plant that may be ingested without risk to man or animals. It may or may not have nutritional properties. Finally, the expression “cactacea plant of at least partially non-assimilable type” means a cactacea plant, at least a part of which is not assimilated by the human (or animal) body during digestion and ends up in the faeces. In one preferred version of the invention, at least 70% by weight of the cactacea plant particles are smaller than 75 μm.

The primary fixing test described later (performed with olive oil), applied to such a preparation based on Opuntia Ficus Indica cladodes, gives fixing ratios (ratio of the mass of fats fixed to the mass of “active” product used—in this case Opuntia, or chitosan for the comparative tests—) ranging between 7 and 19, which are similar (or even higher in the example of fats derived from mayonnaise) to those of chitosan, and very much higher than those of the existing products based on cactus cladodes (the fixing ratios of which range between 2 and 5). It is found that, in the particle size range tested, the ratio obtained increases when the mean particle size of the cactus cladode powder decreases.

This noteworthy result is unexpected for a person skilled in the art. Specifically, cacti consist essentially of water (which they retain) and of fibres, including cellulose, hemicellulose and dietary fibre. Since the chemical properties of fibres generally relate to the length of their chains, it is surprising that the fine comminution of the cactus cladodes, which considerably reduces the length of fibres, improves the fat-fixing property in such spectacular proportions, rather than degrading it as might be expected. This undoubtedly explains why no study has been performed to date regarding the influence of the particle size on the properties of cactus cladodes and why none of the prior art documents provides any teaching regarding the size of the cactus particles of the known powders.

It should moreover be noted that a person skilled in the art might be led to believe that the fat-fixing property of cactus cladodes might be improved by means of hydrophobation of the said fibres (which consists in fixing fatty chains onto the cactus by esterification): however, it is found that such a chemical treatment applied to existing products based on nopal cladodes affords no appreciable effect on the fixing of fats.

The inventors have also shown that the fixing of fats by the cactus cladode powder according to the invention, which is lipophilic, would not give rise to any risk of deficiency of liposoluble vitamins (see Test 5 below).

The cactacea plant is advantageously from the Opuntioidea family, in particular from the genus Opuntia. It is preferably an Opuntia Ficus Indica. It should be pointed out that this species is intensively cultivated in Latin America (especially in Mexico) and sold at low cost. Its cladodes have been consumed for centuries by the native populations, and none of the many studies conducted on this plant has revealed any possible side effects arising from its ingestion.

The cactacea cladodes are preferably chosen when young, and especially less than two years old. Such cladodes have a guaranteed content of soluble and insoluble dietary fibre of greater than 40%, and afford, once prepared according to the invention, a better result as regards the fixing of fats.

Advantageously and according to the invention, the cactacea plant particles are in the form of granules coated with a controlled-release (targeted-release and/or sustained-release) film.

In particular, the cactacea plant particles are in the form of granules coated with an acid-resistant film, especially an enteric film, which is soluble in the intestinal medium. The term “granule” means a particle aggregated of more or less spherical general shape. The in vitro primary fixing tests performed by the inventors have in fact shown that the amount of fats fixed by a cactus powder according to the invention after an incubation in neutral or slightly basic medium (in the case of the intestine) was on average twice as much as that fixed after an incubation in acidic medium (in the case of the stomach). The coating makes it possible to protect the said powder from the gastric juices (medium of pH 1 to 2) and to release it in the intestine (pH close to 7), thus doubling its efficacy (mean fixing ratio of 20).

Advantageously and according to the invention, the cactacea plant particles are in the form of granules coated with a film affording sustained, controlled and gradual release over time. Such a coating allows a gradual release of the powder according to the invention during digestion. As a variant, the cactacea plant particles are incorporated into a water-insoluble matrix comprising, for example, a fatty substance, such as stearic acid, a plastic that is insensitive to the digestive juices and/or a salt or a combination of salts that is (are) sparingly soluble in water, such as calcium sulfate or calcium phosphate, for the purpose of sustained release of the said cactacea plant particles, especially in the intestine.

Advantageously and according to the invention, the coating is both acid-resistant, soluble in the intestine and of sustained-release nature (for a slow, controlled release in the intestine).

The invention also relates to a process for obtaining a preparation having the property of fixing fats, based on cladodes of a cactacea plant of edible and at least partially non-assimilable type, wherein the cactacea plant cladodes are dried and then comminuted so as to obtain a powder of particles, at least 70% by weight of which are smaller than 100 μm (particle size monitored by screening as described above). In one preferred embodiment, the cactacea plant cladodes are comminuted into a powder, at least 70% by weight of the particles of which are smaller than 75 μm. Advantageously and according to the invention, a cactacea plant of the Opuntioidea family, especially of the genus Opuntia and in particular of the species Opuntia Ficus Indica, is used.

Advantageously and according to the invention, the cactacea plant cladodes are sliced into pieces before being dried. The cactacea plant cladodes and/or pieces of cladode are preferably drained in the open air for 12 to 24 hours before being dried. The cactacea plant cladodes and/or pieces of cladode are then dried conventionally in the open air, especially in dryers of the type such as open dryers with racks. As a variant, a static or dynamic dryer maintained at a temperature of between 40 and 80° C. may especially be used to dry the cactacea plant cladodes and/or pieces of cladode. The temperature selected is adapted so as not to bring about conversion of the material by heating it. Advantageously and according to the invention, the cactacea plant cladodes and/or pieces of cladode are dried so as to have a final dryness (ratio of the weight of dry matter to the total weight) of greater than or equal to 90%.

Advantageously and according to the invention, the cactacea plant particles obtained after comminuting the cactacea plant cladodes and/or pieces of cladode are granulated, and the resulting granules are coated with a controlled-release and especially acid-resistant film that is soluble in the intestinal medium and/or a sustained-release film. The term “granulation” (or “granulate”) means the production of bonding bridges between the cactus particles so as to obtain particle aggregates of more or less spherical general shape.

In one advantageous embodiment, the cactacea plant particles are granulated by introducing the said particles into a fluidized-air bed with an air inlet temperature of between 55 and 65° C., and by spraying into the said fluidized-air bed a granulation solution at a pressure of between 2.5 and 3.5 bar. By way of example, in order to granulate about 100 kg of cactus powder according to the invention, the granulation solution is sprayed at a flow rate of between 8 and 12 g/min. The flow rate is adjusted as a function of the desired size of the granules. The aqueous granulation solution preferably used comprises a binder or a combination of binders chosen from the following group: gelatin, gum arabic, xanthan gum, guar gum, carob gum. It should be noted that the granulation makes it possible only to reduce the surface area of the powder to be coated, and thus to reduce the amount of coating solution used, and the duration and cost of the process. The bonding bridges created break instantaneously when the coating dissolves and when the powder is released into aqueous medium. The particle size of the powder thus remains unchanged. The granules obtained after the step of granulating the cactacea plant particles are preferably dried in a fluidized-air bed at an air inlet temperature of between 45 and 55° C.

Advantageously and according to the invention, the granules are coated by introducing the said particles into a fluidized-air bed with an air inlet temperature of between 45 and 55° C., and by spraying into the said fluidized-air bed a coating solution at a pressure of between 2.5 and 3.5 bar, and at a flow rate of between 6 and 10 g/min if it is desired to coat about 100 kg of granules.

To produce an acid-resistant coating that is soluble in the intestine, the coating solution preferably used comprises a solvent such as ethanol and a film-forming agent or a combination of film-forming agents chosen from the following group: cellulose acetophthalate, carboxymethylcellulose (CMC). The coating solution also preferably comprises a plasticizer or a combination of plasticizers chosen from the following group: triacetin, polyethylene glycol (PEG). The resulting film is resistant to acidic pH and soluble at neutral or slightly basic pH. The cactus powder ingested in the form of coated granules is thus released when the granules pass through the intestine.

To produce a sustained-release coating, the coating solution preferably used comprises a film-forming agent or a combination of film-forming agents that show slow dissolution in water, chosen from the following group: polyvinyl acetate, shellac. As a variant, a coating solution comprising a film-forming agent or a combination of film-forming agents that is (are) insoluble in water but semi-permeable, such as a polymer of the gum arabic type, is used. These agents that are insoluble (but semi-permeable) or sparingly soluble in water may be combined with acid-resistant film-forming agents that are soluble in the intestine, in order to allow a sustained release of the cactus powder only in the intestine.

As a variant, the cactacea plant particles obtained after comminuting the cactacea plant cladodes and/or pieces of cladode are incorporated into an insoluble matrix so as to allow a controlled-release of the said particles when they are ingested. Such an insoluble matrix comprises, for example, a fatty substance, such as stearic acid, and a plastic that is insensitive to the digestive juices and/or a sparingly water-soluble salt, such as calcium sulfate or calcium phosphate.

The invention covers a preparation and a process for obtaining such a preparation, characterized in combination by some or all of the characteristics mentioned hereinabove and hereinbelow.

The invention also covers the use of cactacea plant cladodes dried and comminuted into a powder of particles, at least 70% by weight of which are smaller than 100 μm, to obtain a preparation intended to be ingested in combination with a food ration in order to fix in vivo the fats present in the said food ration.

In particular, the invention relates to the use of cactacea plant cladodes dried and comminuted into a powder of particles, at least 70% by weight of which are smaller than 100 μm, to obtain a therapeutic composition for treating obesity. The invention also relates to a method for fixing fats in vivo and especially for treating obesity, according to which a cactacea-based preparation as claimed is administered orally to the patient.

The invention also covers a therapeutic composition comprising a cactacea-based preparation as claimed, and a pharmaceutically acceptable excipient. Such a therapeutic composition is advantageously conditioned in a presentation form that is suitable for oral administration. It should be noted that the therapeutic composition may contain other active principles that are compatible with the cactacea-based preparation according to the invention.

The cactacea plant cladode powder may be conditioned alone, in the form of tablets, gel capsules, soft or rigid capsules or quite simply a powder to be mixed into a drink (water or other soda, fruit juice, etc.) or to be sprinkled onto food. It is then preferably ingested during a meal. As a variant, it may be incorporated into food.

When mixed with non-fatty food, it makes it possible to fix the fats of fatty foods ingested during the same meal. It should be noted that the cactus cladodes intended to be mixed with a liquid (soda, etc.) are preferably comminuted into a particle size of less than 20 μm. When mixed with fatty food, the powder makes it possible to fix the fats thereof before (in vitro) and/or after. (in vivo) ingestion of the said food. In this case, the powder should have a particle size that is adapted such that the cactus particles and the possible fats fixed to these particles are not perceptible in the mouth when the food is consumed.

The invention also covers a food composition—simple or mixed food—and/or a food-supplement comprising a cactacea-based preparation as claimed. The term “food supplement” means concentrated sources of nutrients or other substances having a nutritional or physiological role, alone or in combination, marketed in unit dose form, and the object of which is to supplement the normal food intake. The food supplements satisfy strict legislative regulations in terms of composition, dosage, labelling, etc. The invention also relates to the use of a cactacea-based preparation as claimed, to obtain a food composition and/or a food supplement.

The description that follows gives a few non-limiting examples of the production according to the invention of a cactacea-based preparation, and also a summary of the efficacy studies performed on various products including preparations according to the invention.

Test 1:

The propensity of a cactus cladode powder to fix fats was checked using a device simulating the functioning of the human digestive apparatus, in order to be able to extend the results of the primary fixing tests to the particular case of digestion. The device used is described in U.S. Pat. No. 5,525,305. It reproduces the successive dynamic conditions of the digestive apparatus, such as the pH variations between the stomach and the small intestine, the concentrations of (pro)enzymes, juices, salts and other secretions of the various organs concerned (gastric juice with enzymes, electrolytes, bile, pancreatin, etc.) and the kinetics of transit of the chyme through the stomach and the intestines. This device makes it possible to study the digestibility and the availability for assimilation of nutrients, and also the stability of specific ingredients (active proteins, peptides, etc.). This device was validated by means of in vivo experiments.

2 g of Opuntia Ficus Indica are mixed, homogeneously, into a meal composed of 20 g of sunflower oil and 144 g of fat-free yoghurt. A control preparation that is identical (oil+yoghurt) but comprises no Opuntia is also produced and ingested in the simulator during a subsequent control experiment. The simulator is set up to reproduce the digestive apparatus of a healthy young adult, after ingestion of semi-liquid food. The foods ingested remain in the simulator for four hours. A specific system is moreover used to isolate the digested and assimilated products from the device.

The following are collected and analysed: the residual materials in the stomach-and the duodenum (as simulated by the device) at the end of the experiment, the residual materials in the small intestine at the end of the experiment, samples of fractions assimilated after each of the four hours of experiment (a portion of the said samples is moreover mixed together, to form a sample representing a fraction of the total amount of assimilated foods). The volumes of the various samples taken are measured, and half the amount is stored at −20° C. for a double analysis.

The composition of the fatty acids in the assimilated fractions is analysed according to the following process. The samples are hydrolysed in alkaline medium for one hour at 70° C. After cooling and acidifying, an internal standard is added and the fatty acids are extracted using a hexane bath. After evaporating the hexane phase, methyl esters are prepared from the said fatty acids, using a BF₃/methanol reagent. After cooling, a solution of hexane and of saturated sodium chloride is added. After stirring, the upper hexane phase is taken, diluted and subjected to a gas chromatographic analysis.

The results are given in Table 1. It should be noted that the amount of triglycerides initially present in each of the ingested nutritional preparations (control preparation and preparation with Opuntia) is 985.5 mg/g of oil, i.e. in total 19.7 g of triglycerides ingested per nutritional preparation.

TABLE 1

| | |
|---|---|
| Control | Preparation |
| preparation | with Opuntia |

| | | |
|---|---|---|
| Total amount of fatty | 9.40 ± 0.34 g (1) | 6.74 ± 0.38 g (2) |
| acids assimilated by the | | |
| digestive apparatus: | | |
| Percentage of fatty acids | 90 ± 9% | 71 ± 7% |
| assimilated: | | |

| | |
|---|---|
| Total amount of fatty | 2.64 ± 0.36 g (3) |
| acids fixed by the | |
| Opuntia (3) = (1) − (2): | |
| Fatty acid fixing ratio*: | 1.32 ± 0.18 |

*In this example, the fixing ratio is equal to the weight of fatty acids (rather than of oil) fixed by the Opuntia to the weight of Opuntia used.

The ability of the cactus powder (in this instance Opuntia Ficus Indica) to fix the fats is thus confirmed in the particular case (from a chemical and kinetic viewpoint) of digestion.

The distribution of the various fatty acids assimilated is given in Table 2 for the two preparations (control and with Opuntia).

TABLE 2

| Fatty acids | Control | Preparation with |
|---|---|---|
| assimilated | preparation (mg) | Opuntia (mg) |
| C14 | 14.0 ± 4.1 | 9.6 ± 0.09 |
| C16 | 679.8 ± 43.0 | 496.1 ± 38.9 |
| C18 | 354.8 ± 6.6 | 260.2 ± 7.3 |
| C18: 1 c | 2210.3 ± 120.8 | 1565.9 ± 67.9 |
| C18: 2 c | 5368.0 ± 233.8 | 3787.0 ± 158.8 |
| C20: 4 c | 94.0 ± 14.4 | 72.2 ± 13.8 |
| Not identified | 676.3 ± 79.9 | 548.6 ± 91.5 |
| Total | 9397.2 ± 342.0 | 6739.6 ± 378.0 |

It is found that the ratio between the amount of fatty acids assimilated in the control experiment and the amount assimilated in the experiment with Opuntia varies little as a function of the fatty acid. The fixing of the fatty acids with the cactus powder is therefore unselective.

Procedure of the Primary Fixing Test

Materials:

Oven set at 37° C.

Balance accurate to 1 mg

250 ml-beaker

85 ml-stoppered glass centrifuge tubes (dimensions 44×98 mm)

100 μl and 1000 μl micropipettes

Centrifuge set at 2000 rpm (670g)

Reagents:

0.1N hydrochloric acid

| | | |
|---|---|---|
| Buffer: | tris(hydroxymethyl)aminomethane acid | 120 g |
| | 35% HCl | 49 ml |
| | water qs | 200 ml |

Three test tubes containing 25 ml of 0.1N HCl are prepared. 6 g (mass M) of oil are added to the first tube, 12 g (M) of melted butter in the second, and 12 g (M) of mayonnaise in the third. 0.6 g (mass M1) of the test preparation (commercially available cactus preparation, cactus powder according to the invention, chitosan, etc.) are added to each of the tubes. The solutions obtained are mixed vigorously by shaking the tube by hand for 30 seconds, and are placed in an oven at 37° C. and incubated for two hours. 5 ml of a pH 7.3 buffer solution are then added to each of the tubes. After a further manual shaking, the tubes are returned to the oven at 37° C. for three hours. They are then centrifuged for five minutes at 2000 rpm. The supernatant fat is then removed and weighed (mass M2), and the ratio of the mass of fatty substance (oil, mayonnaise or butter) fixed by the preparation (M-M2) to the mass of preparation used (M), referred to as the fixing ratio, is calculated. For each test preparation, the test is repeated several times to ensure the validity of the results.

Test 2: Comparison of the Fixing Capacities of a Number of Cactus-Based Commercial Products, of Three Preparations According to the Invention and of Chitosan-Based Commercial Products

Various commercially available products and three preparations according to the invention were tested according to the protocol of the primary fixing test. The results are given in Table 3 below.

TABLE 3

| | | Frac- | Fixing ratio** | | |
|---|---|---|---|---|---|
| Preparation (packaging + supplier | | tion < | | Mayon- | |
| or registered trademark) | | 75* | Oil | naise | Butter |
| Commercially available cactus | | | | | |
| preparations: | | | | | |
| 1 | Nopal powder, dietary | 19% | 2 ± 2 | 8 ± 3 | 5 ± 2 |
| | supplement for diabetics (acts | | | | |
| | especially on glycaemia)- | | | | |
| | PROESA Std | | | | |
| 2 | Nopal powder for diabetics - | 48% | 1 ± 2 | 13 ± 3 | 4 ± 2 |
| | PLANTA MEX 60 M | | | | |
| 3 | Nopal powder according to | 87% | 8 ± 2 | 20 ± 3 | 10 ± 2 |
| | the invention (batch | | | | |
| | ABBN0075) | | | | |
| 4 | Nopal powder according to | 80% | 7 ± 2 | 17 ± 3 | 11 ± 2 |
| | the invention (batch | | | | |
| | BER001) | | | | |
| 5 | Nopal powder according to | 75% | 10 ± 2 | 17 ± 3 | 11 ± 2 |
| | the invention (batch SM6041) | | | | |
| 6 | BioCaptol ®, chitosan, | nd | 6 ± 2 | 10 ± 3 | 8 ± 2 |
| | slimming diet supplement for | | | | |
| | fixing fats | | | | |
| 7 | UltimateNutrition ®, | nd | 9 ± 2 | 13 ± 3 | 11 ± 2 |
| | chitosan, for fixing fats | | | | |
| 8 | Absorbitol ®, chitosan, | nd | 9 ± 2 | 12 ± 3 | 10 ± 2 |
| | for fixing fats | | | | |

*Fraction < 75: fraction of particles smaller than 75 μm, controlled by screening.
**Fixing ratio = g of oil or mayonnaise or butter fixed/g of cactus powder or g of chitosan.

The powders prepared according to the invention (samples 3 to 5) thus have fixing ratios that are considerably higher than those of the commercial nopal powders. The process for preparing these powders is therefore in direct relationship with the capacity of the product to fix fats. Moreover, the powders according to the invention are of comparable or even significantly higher efficacy relative to the chitosan-based products (samples 6 to 8).

Test 3: Influence of the Particle Size

A particle size profile is produced for a finely comminuted Opuntia Ficus Indica cladode powder (according to the invention). This profile is produced according to the NF standard ISO 2591-1 (control screening) by superimposing four screens with mesh apertures of 150, 106, 75 and 53 μm (square mesh holes). The screening results are given in Table 4. Each of the fractions is collected in order to test its fat-fixing capacity.

TABLE 4

| | Retainings | | | |
|---|---|---|---|---|
| | on each | | | |
| Particle sizes | screen | | Nominal mesh | Cumulative passing |
| d (μm) | grams | % | aperture (μm) | matter (%) |
| d > 150 | 2.57 | 10.3 | 150 | 89.7 |
| 150 ≧ d > 106 | 1.62 | 6.5 | 106 | 83.2 |
| 106 ≧ d > 75 | 1.92 | 7.7 | 75 | 75.5 |
| 75 ≧ d > 53 | 2.44 | 9.8 | 53 | 65.6 |
| 53 ≧ d | 16.34 | 65.6 | 0 | 0 |
| Total | 24.89 | 100 | | |

The capacity to fix oil, mayonnaise and butter (primary fixing test) of the unscreened powder and of each of the preceding isolated fractions is then measured. The results are given in Table 5.

TABLE 5

Fixing ratio:

| Opuntia fraction tested: | Oil | Mayonnaise | Butter |
|---|---|---|---|
| Unscreened powder | 10 ± 2 | 19 ± 3 | 12 ± 2 |
| d > 150 μm | 4 ± 2 | 12 ± 3 | 8 ± 2 |
| 150 μm ≧ d > 106 μm | 5 ± 2 | 13 ± 3 | 8 ± 2 |
| 106 μm ≧ d > 75 μm | 5 ± 2 | 13 ± 3 | 9 ± 2 |
| 75 μm ≧ d > 53 μm | 7 ± 2 | 15 ± 3 | 13 ± 2 |
| 53 μm ≦ d | 10 ± 2 | 19 ± 3 | 13 ± 2 |

Since the above samples are obtained from the same powder, it may be concluded with certainty from test 3 that the oil-fixing capacity of a cactus powder is a decreasing function of the particle size (in the particle size range tested).

Test 4: Influence of the pH

The object of this test is to compare the fat-fixing capacity of the same Opuntia Ficus Indica powder, prepared according to the invention, as a function of the pH of the incubation medium. A first test is performed under the conditions of the primary fixing test already described, with 0.3 g of test sample. A second test is performed by replacing the 25 ml of 0.1N HCl of the preceding test with 25 ml of water buffered to pH 7±0.1. The results are given in Table 6.

TABLE 6

| Test protocol | Fixing ratio: |
|---|---|
| Primary fixing test with | 11 ± 2 |
| incubation at gastric pH (HCl) | |
| Modified primary test with | 20 ± 2 |
| incubation at intestinal pH (H₂O) | |

A doubling of the fat-fixing capacity of the Opuntia powder is observed if the incubation takes place in a neutral medium, such as the intestine. The incubation at gastric pH therefore greatly limits the efficacy of the cactus powder. This is why the invention advantageously envisages protecting the cactus powder with an acid-resistant intestinal-release coating.

Test 5: Fixing of Liposoluble Vitamins with a Cactus Powder According to the Invention

The test consists in placing a fat-fixing agent in contact with a fatty substance (sunflower oil) containing approximately the daily Recommended Dietary Intake (RDI) of vitamins A and E, under conditions (aqueous phase, temperature, pH) similar to those of digestion. The fat-fixing agent forms a gel with the fatty substance and reduces the amount of supernatant fat in the medium. Analysis of the amount of vitamins in the supernatant fatty phase without and with fat-fixing agent makes it possible to evaluate the capacity of this agent to specifically fix the liposoluble vitamins.

Materials:

Oven set at 37° C.

Balance accurate to 1 mg

Standard laboratory glassware

Magnetic stirrer-hotplate

85 ml-stoppered glass centrifuge tubes (dimensions 44×98 mm)

100 μl- and 1000 μl-micropipettes

Centrifuge set at 2000 rpm (670 g)

Reagents:

Vitamins A and E (supplied by Sigma-Aldrich-Fluka Produits Chimiques)

0.1N hydrochloric acid

Commercial sunflower oil (with guaranteed vitamin E content)

pH 7.3 buffer: tris(hydroxymethyl)aminomethane acid 121.0 g

| | |
|---|---|
| 35% HCl | 49.0 ml |
| water qs | 200 ml |

Amounts of vitamins A and E are added to the commercial sunflower oil such that 20 g of oil thus enhanced, referred to as “standardized oil”, contain approximately the RDI for a female adult (i.e. vitamin A: 800 μg, vitamin E: 12 mg).

25 ml of 0.1N HCl are poured into each of the two centrifuge tubes, and 20 g of standardized oil and 0.6 g of Opuntia powder according to the invention are added thereto. Each stoppered tube is shaken vigorously for 30 seconds and is then incubated for two hours in the oven at 37° C. 5 ml of pH 7.3 buffer solution are added to each of the tubes, which are re-stoppered, shaken vigorously for 30 seconds and reincubated in the oven at 37° C. for three hours. Each tube is subjected to centrifugation at 2000 rpm for five minutes. The tubes are then maintained at 37° C., while the supernatant fat is removed. The vitamin A and E content of the removed supernatant is analysed.

The above experiment is repeated, replacing the mass of Opuntia with 0.6 g of distilled water, in order to perform a control. The vitamin A and E contents in the supernatant fat at the end of the experiment in the test (in the presence of Opuntia powder according to the invention) and in the control (distilled water) are compared. The results are given in Table 7 below.

TABLE 7

| | Liposoluble vitamins | | Capacity (C)* |
|---|---|---|---|
| | concentration of the | | for specific |
| | supernatant oil at the end | | fixing of the |
| | of the experiment (per 100 g | | vitamins with |
| | of oil) | | the powder |
| | Control | | according to |
| | (V_{control}) | Test (Vₜₑₛₜ) | the invention |
| Vitamin A | 4.65 mg/100 g | 4.40 mg/100 g | 5.4% |
| (retinol) | | | |
| Vitamin E | 58.6 mg/100 g | 57.5 mg/100 g | 1.9% |
| (di-α-tocopherol) | | | |

*Capacity for specific fixing of the vitamins by the Opuntia powder according to the invention: C = 1 − (Vₜₑₛₜ/V_{control})

The concentration differences observed between the control and the test are small. A statistical study would need to be performed to determine whether they are effectively due to the Opuntia powder according to the invention, or whether they result simply from the experimental procedure. In any case, the differences found are not significant and prove that the Opuntia powder according to the invention does not fix the liposoluble vitamins A and E specifically.

### EXAMPLE 6

Coating

A powder according to the invention is granulated in a fluidized-air bed having the following parameters:

| | |
|---|---|
| Air inlet temperature: | 60° C. |
| Vaporization flow rate: | 10 g/min |
| Spray pressure: | 3 bar |
| Granulation solution: | 600 ml of H₂O |
| | 40 g of gelatin 30 Bloom |

The Opuntia powder is preheated so as to reach a temperature of 35° C. The granulation solution is then sprayed onto the powder moving in the air bed, preferably opposite the air distributor (top spraying), or in the mid-section of the device, perpendicularly to the air bed. The composition of the granulation solution and the amount vaporized are adjusted as a function of the desired final particle size, which depends especially on the final conditioning-of the cactus powder (tablets, gel capsules, rigid or soft capsules, dietary powder, etc.).

Once the desired particle size has been reached, the powder is dried in the fluidized-air bed, the temperature of which is adjusted to 50° C. The collected granules are then screened, the excessively large granules being comminuted again and reintroduced into the process, the granules of insufficient size being recycled during another step of constructive granulation.

The granules are then coated in the fluidized-air bed, the parameters of which are as follows:

| | |
|---|---|
| Air inlet temperature: | 50° C. |
| Vaporization flow rate: | 8 g/min. |
| Spray pressure: | 3 bar |
| Coating solution: | 500 g of cellulose acetophthalate |
| | 150 g of triacetin |
| | 2 l of ethanol |
| | 8 l of methylene chloride |

The granules are heated so as to reach a temperature of 35° C. The coating solution is sprayed into the fluidized-air bed (for example by top-spraying) under the abovementioned conditions. After coating, the granules are dried in the fluidized-air bed at a temperature of 50° C.

Test 7: Properties of the Coating of Example 6

The coating is first tested in acidic medium according to the following protocol. A solution is prepared comprising:

52 ml of 37% HCl,

120 g of Tris hydroxymethyl aminomethane buffer sufficient quantity of water for 200 ml of solution,

to which are added 2 g of nopal powder premixed with the green colouring agent E131, and coated according to Example 6. The colouring agent makes it possible to monitor the release of the nopal into the medium. After manual shaking of the solution, a reading of the said solution is taken at 420 nm using a spectrophotometer. No coloration appears in the solution: the coating produced is resistant to very acidic pH (approximate pH of the solution: 2-3); it is therefore acid-resistant.

A control solution is prepared comprising the abovementioned compounds, to which are added 2 g of nopal powder premixed with the colouring agent E131, but not coated. According to the results of the spectrophotometer, the colouring agent migrated into the solution.

The coating is then tested in basic medium. To do this, a solution is prepared comprising:

46 ml of 37% HCl,

120 g of Tris hydroxymethyl aminomethane buffer

a sufficient quantity of water for 200 ml of solution,

to which are added 2 g of nopal powder premixed with the green colouring agent E131, and coated according to Example 6. After shaking the solution, a reading of the said solution is taken at 420 nm using a spectrophotometer. The release of the green dye into the medium is detected: the coating produced is soluble at basic pH (approximate pH of the solution: 8-9); it therefore becomes released in the intestine.

1. Preparation having the property of fixing fats, based on cladodes of a cactacea plant of edible and at least partially non-assimilable type, wherein the cactacea plant is in the form of a powder of particles, at least 70% by weight of which are smaller than 100 μm.

2. Preparation according to claim 1, wherein at least 70% by weight of the cactacea plant particles are smaller than 75 μm.

3. Preparation according to either of claims 1 and 2, wherein the cactacea plant is of the Opuntioidea family, in particular of the genus Opuntia and especially of the species Opuntia Ficus Indica.

4. Preparation according to one of claims 1, 2 and 3, wherein the cactacea plant cladodes are less than two years old.

5. Preparation according to one of claims 1 to 4, wherein the cactacea plant particles are in the form of granules coated with an acid-resistant film that is soluble in the intestinal medium.

6. Preparation according to one of claims 1 to 5, wherein the cactacea plant particles are in the form of granules coated with a sustained-release film.

7. Preparation according to one of claims 1 to 5, wherein the cactacea plant particles are incorporated into a water-insoluble matrix comprising a fatty substance, such as stearic acid, a plastic that is insensitive to the digestive juices and/or a salt, or a combination of salts, which is (are) sparingly soluble in water, such as calcium sulfate or calcium phosphate.

8. Process for obtaining a preparation having the property of fixing fats, based on cladodes of a cactacea plant of edible and at least partially non-assimilable type, wherein the cactacea plant cladodes are dried and then comminuted so as to obtain a powder of particles, at least 70% by weight of which are smaller than 100 μm.

9. Process according to claim 8, wherein the cactacea plant cladodes are comminuted into a powder, at least 70% by weight of the particles of which are smaller than 75 μm.

10. Process according to either of claims 8 and 9, wherein a cactacea plant of the family Opuntioidea, in particular of the genus Opuntia and especially of the species Opuntia Ficus Indica, is used.

11. Process according to one of claims 8, 9 and 10, wherein cactacea plant cladodes less than two years old are used.

12. Process according to one of claims 8 to 11, wherein the cactacea plant cladodes are sliced into pieces before being dried.

13. Process according to one of claims 8 to 12, wherein the cactacea plant cladodes and/or cladode pieces are drained in the open air for 12 to 24 hours before being dried.

14. Process according to one of claims 8 to 13, wherein the cactacea plant cladodes and/or cladode pieces are conventionally dried in the open air.

15. Process according to one of claims 8 to 14, wherein a static or dynamic dryer maintained at a temperature of between 40 and 80° C. is used to dry the cactacea plant cladodes and/or cladode pieces.

16. Process according to one of claims 8 to 15, wherein the cactacea plant cladodes and/or cladode pieces are dried so as to have a final dryness of greater than or equal to 90%.

17. Process according to one of claims 8 to 16, wherein the cactacea plant particles obtained are incorporated into a water-insoluble matrix comprising a fatty substance, such as stearic acid, and a plastic that is insensitive to the digestive juices and/or a salt, or a combination of salts, that is (are) sparingly soluble in water, chosen from the following group: calcium sulfate, calcium phosphate.

18. Process according to one of claims 8 to 16, wherein the cactacea plant particles obtained are granulated, and in that the granules are coated with an acid-resistant film that is soluble in the intestine and/or that is a sustained-release film.

19. Process according to claim 18, wherein the cactacea plant particles are granulated by introducing the said particles into a fluidized-air bed with an air inlet temperature of between 55 and 65° C., and by spraying into the said fluidized-air bed a granulation solution at a pressure of between 2.5 and 3.5 bar.

20. Process according to claim 19, wherein an aqueous granulation solution is used comprising a binder or a combination of binders chosen from the following group: gelatin, gum arabic, xanthan gum, guar gum, carob gum.

21. Process according to one of claims 18, 19 and 20, wherein the granules obtained after the step of granulating the cactacea plant particles are dried in a fluidized-air bed with an air inlet temperature of between 45 and 55° C.

22. Process according to one of claims 18 to 21, wherein the granules are coated by introducing the said granules into a fluidized-air bed with an air inlet temperature of between 45 and 55° C., and by spraying into the said fluidized-air bed a coating solution at a pressure of between 2.5 and 3.5 bar.

23. Process according to claim 22, wherein a coating solution is used comprising a film-forming agent or a combination of film-forming agents that is (are) acid-resistant and soluble in the intestinal medium, and/or that show slow dissolution in water or are insoluble in water but semi-permeable, chosen from the following group: cellulose acetophthalate, carboxymethylcellulose, polyvinyl acetate, shellac, polymer of the gum arabic type.

24. Process according to claim 23, wherein a coating solution is used comprising a plasticizer or a combination of plasticizers chosen from the following group: triacetin, polyethylene glycol.

25. Use of cactacea plant cladodes dried and comminuted into a powder of particles, at least 70% by weight of which are smaller than 100 μm, to obtain a preparation intended to be ingested in combination with a food ration in order to fix in vivo the fats present in the said food ration.

26. Use of cactacea plant cladodes dried and comminuted into a powder of particles, at least 70% by weight of which are smaller than 100 μm, to obtain a therapeutic composition for treating obesity.

27. Therapeutic composition, wherein it comprises a preparation according to one of claims 1 to 7 and a pharmaceutically acceptable excipient.

28. Therapeutic composition according to claim 29, in a presentation form that is suitable for oral administration.

29. Food composition, wherein it comprises a preparation according to one of claims 1 to 7.

30. Food supplement, wherein it comprises a preparation according to one of claims 1 to 7.
